(19) United States
(12) Patent Application Publication
(10) Pub. No.: US 2004/0225190 A1
(43) Pub. Date: Nov. 11, 2004

(54) Capsule endoscope and a capsule endoscope system

(75) Inventors: Seiichiro Kimoto, Tokyo (JP); Noriyuki Fujimori, Tokyo (JP); Hiroshi Suzushima, Tokyo (JP); Toshiaki Shigemori, Tokyo (JP); Tsutomu Nakamura, Tokyo (JP); Ayako Nagase, Tokyo (JP); Tetsuo Minai, Tokyo (JP); Hatsuo Shimizu, Tokyo (JP); Takemitsu Honda, Tokyo (JP); Katsuyoshi Sasagawa, Tokyo (JP); Katsuya Suzuki, Tokyo (JP); Masayuki Hashimoto, Tokyo (JP); Tatsuya Orihara, Tokyo (JP); Kazutaka Nakatsuchi, Tokyo (JP)

Correspondence Address: SCULLY SCOTT MURPHY & PRESSER, PC, 400 GARDEN CITY PLAZA, GARDEN CITY, NY, 11530

(73) Assignee: OLYMPUS CORPORATION, TOKYO (JP)

(21) Appl. No.: 10/830,774

(22) Filed: Apr. 23, 2004

(30) Foreign Application Priority Data
Apr. 25, 2003 (JP) 2003-122806

(51) Int. Cl. A61B 1/06

(52) U.S. Cl. 600/177; 600/160

(57) ABSTRACT

A capsule endoscope has a front cover having a window for illuminating light and a window for capturing images. The window for illuminating light is flat. The window for capturing images is flat or convex. Because the window for illuminating light is flat, the light passes through the window without being reflected toward an image capturing unit.

### BACKGROUND OF THE INVENTION

1) Field of the Invention

The present invention relates to swallowable and capsule-shaped endoscopes and a capsule endoscope system.

2) Description of the Related Art

Swallowable and capsule-shaped endoscopes are known in the art. A patient swallows such a capsule endoscope and as the capsule endoscope passes through an abdominal cavity, it captures images of a stomach, intestines etc. An example of such a capsule endoscope is described below with reference to FIG. 7.

As shown in FIG. 7, the conventional capsule endoscope includes a watertight capsule casing 8 having a front cover 5. The capsule casing 8 houses an image capturing unit 1 that captures images of an inside the abdominal cavity, an illuminating unit 2 that outputs a light to illuminate the inside of the abdominal cavity, a power-supply unit 3 that supplies power to the image capturing unit 1 and the illuminating unit 2.

The illuminating unit 2 and the image capturing unit 1 are disposed near each other and they face toward a front side through the front cover 5. The front cover 5 is partly or fully transparent so that when the illuminating unit 2 outputs the light, the light illuminates the inside of the abdominal cavity and the image capturing unit 1 can capture images of inside of the abdominal cavity.

The front cover 5 is semispherical, because, it is easier for a patient to swallow the capsule endoscope if the front cover 5 is semispherical, and body fluids can not remain on the front cover 5 if the front cover 5 is semispherical (see Japanese Patent Application Laid-open Publication No. 2001-95756).

However, because the front cover 5 is dome-shaped, a window 5a, for passing the light, in the front cover 5 has a curvature as shown in FIG. 8. Therefore, the light L output by the illuminating unit 2 does not necessarily pass through the window 5a at right angles to the window 5a so that some part of the light L reflects back from the window 5a in the form of a reflected light RL. The reflected light RL enters the image capturing unit 1 and degrades the image quality.

### SUMMARY OF THE INVENTION

It is an object of the present invention to solve at least the problems in the conventional technology.

A capsule endoscope according to an aspect of the present invention includes an image capturing unit that has an image capturing section which can capture images of an inside of an abdominal cavity; an illuminating unit that outputs light and illuminates the inside of the abdominal cavity with the light; a power-supply unit that supplies power to the image capturing unit and the illuminating unit; a front cover that covers the image capturing unit and the illuminating unit and is provided with an illuminating window that lets the light output by the illuminating unit to pass through and that makes it hard for the light output by the illuminating unit to be reflected toward the image capturing unit; and a capsule casing that is attached to the front cover such that there is a watertight space inside, wherein the image capturing unit, the illuminating unit, and the power-supply unit are housed in the watertight space.

A capsule endoscope system according to another aspect of the present invention includes a capsule endoscope including an image capturing unit that has an image capturing section which can capture images of an inside of an abdominal cavity; an illuminating unit that outputs light and illuminates the inside of the abdominal cavity with the light; a power-supply unit that supplies power to the image capturing unit and the illuminating unit; a front cover that covers the image capturing unit and the illuminating unit and is provided with an illuminating window that lets the light output by the illuminating unit to pass through and that makes it hard for the light output by the illuminating unit to be reflected toward the image capturing unit; a communication unit that transmits to outside image information acquired by the image capturing unit; and a capsule casing that is attached to the front cover such that there is a watertight space inside, wherein the image capturing unit, the illuminating unit, and the power-supply unit are housed in the watertight space; a package that covers the capsule endoscope before the capsule endoscope is used; a receiving unit that receives the image information from the capsule endoscope; and an information processor that processes the image information received by the receiving unit.

A capsule endoscope according to still another aspect of the present invention includes a casing to house an illuminating unit and an image capturing unit, and a cover for the casing, the cover having an illuminating window that has a flat surface and through which the illuminating unit illuminates a portion inside a body of a patient; and an image capturing window through which the image capturing unit captures an image of the portion illuminated by the illuminating unit.

The other objects, features, and advantages of the present invention are specifically set forth in or will become apparent from the following detailed description of the invention when read in conjunction with the accompanying drawings.

### BRIEF DESCRIPTION OF THE DRAWINGS

FIG. 1 is a schematic diagram of a capsule endoscope according to an embodiment of the present invention;

FIG. 2 is a schematic diagram of a capsule endoscope system;

FIG. 3 is a schematic diagram of a capsule endoscope according to another embodiment of the present invention;

FIG. 4 is a perspective view of a front cover of the capsule endoscope;

FIG. 5 is a schematic diagram for explaining a light output by an illuminating unit of the capsule endoscope;

FIG. 6 is a schematic diagram for explaining of a reflection of the light output by the illuminating unit;

FIG. 7 is a schematic diagram of a conventional capsule endoscope; and

FIG. 8 is a schematic diagram for explaining the problems in the conventional capsule endoscope.

### DETAILED DESCRIPTION

Exemplary embodiments of a capsule endoscope and a capsule endoscope system according to the present invention are described below in detail with reference to the accompanying drawings.

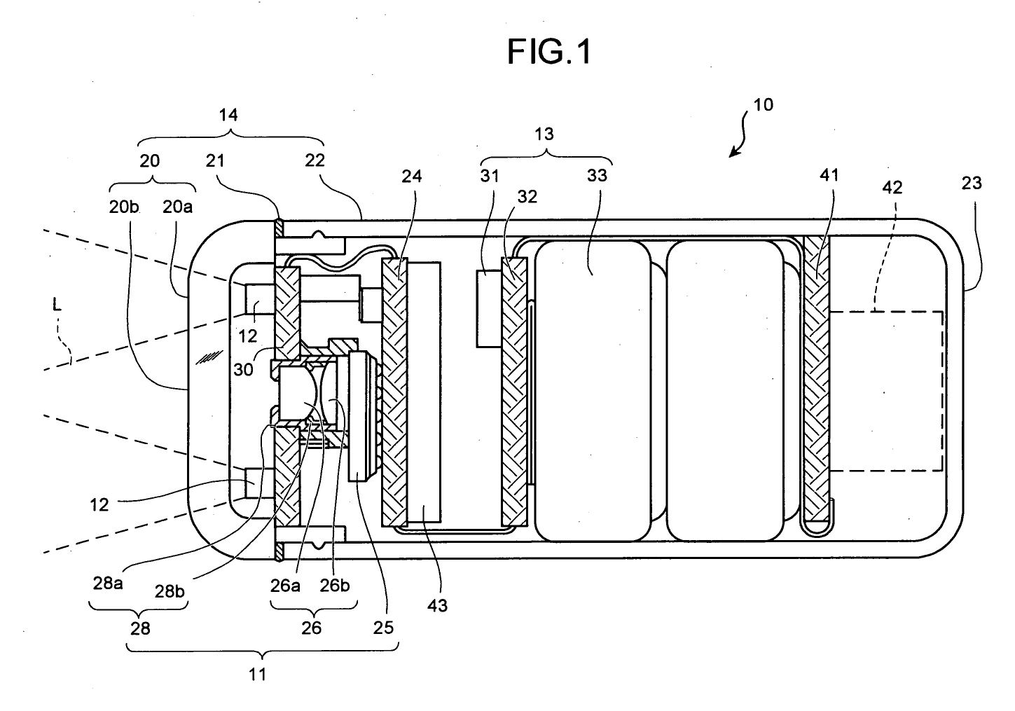

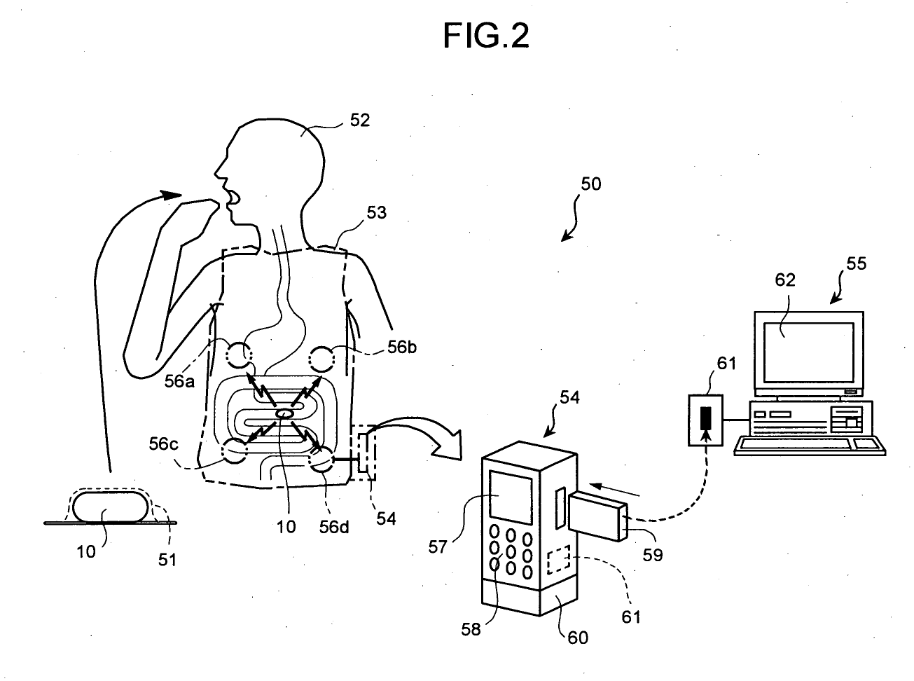

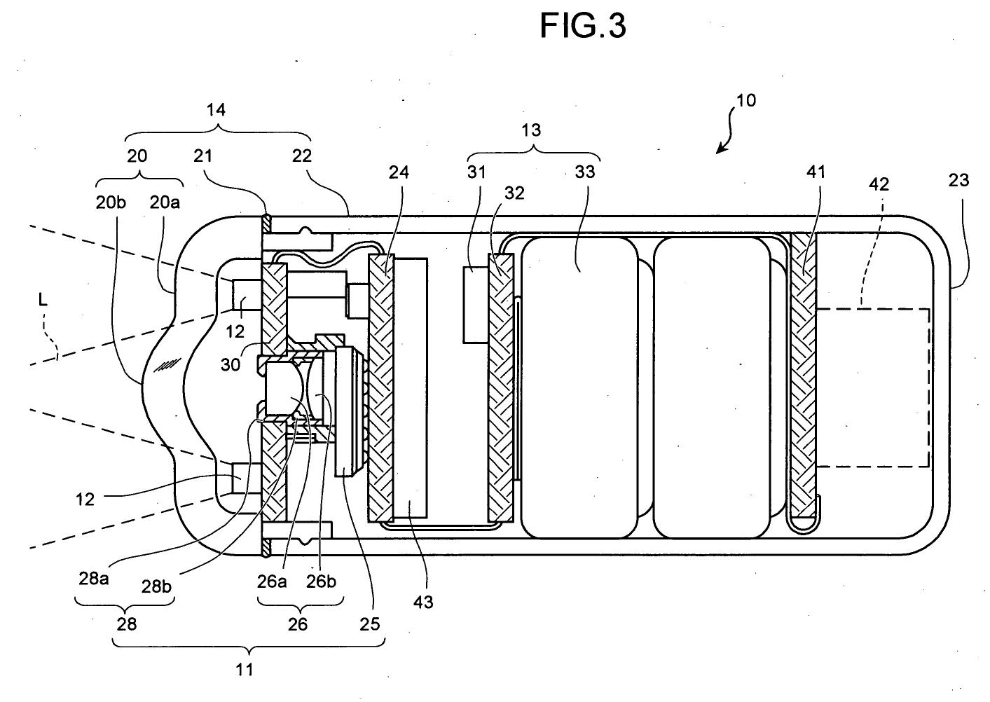

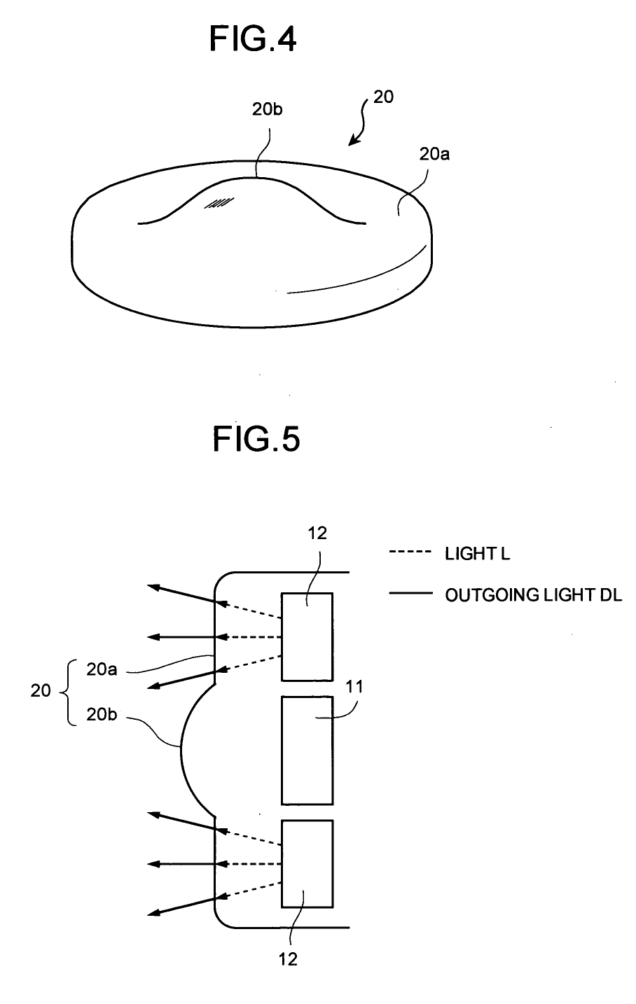

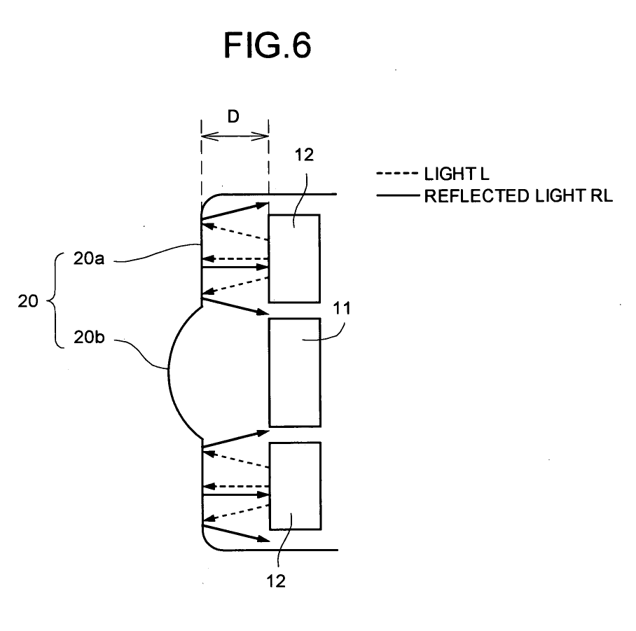

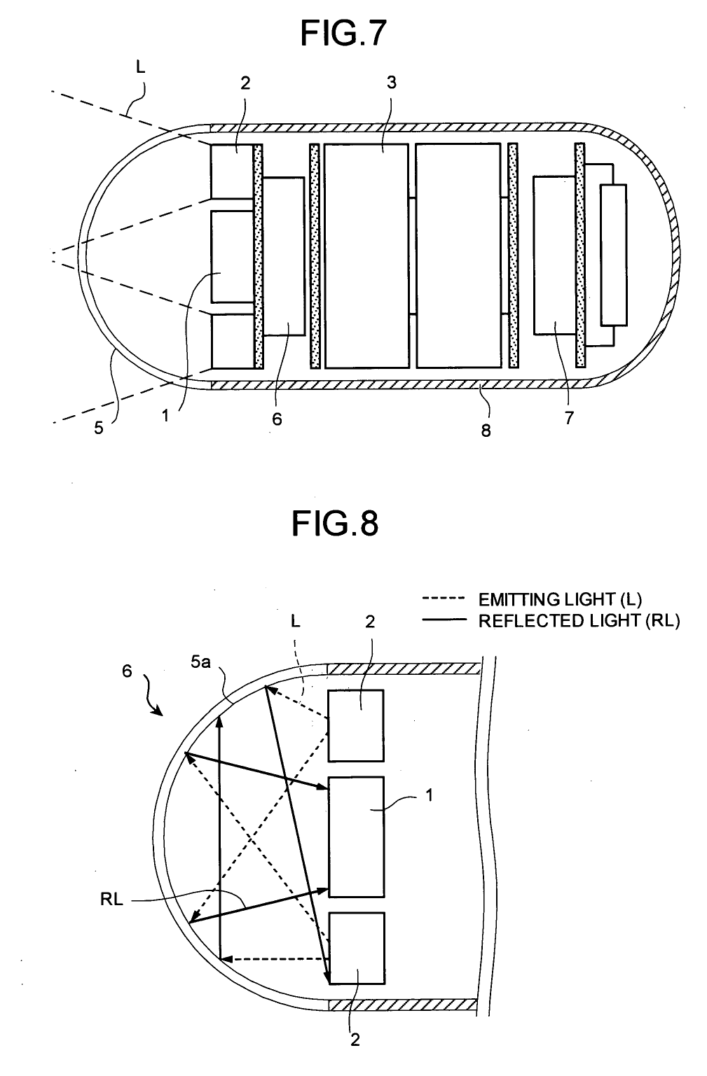

FIG. 1 is a schematic diagram of a capsule endoscope according to an embodiment of the present invention. FIG. 2 is a schematic diagram of a capsule endoscope system. FIG. 3 is a schematic diagram of a capsule endoscope according to another embodiment of the present invention. FIG. 4 is a perspective view of a front cover of the capsule endoscope. FIG. 5 is a schematic diagram for explaining a light output by an illuminating unit of the capsule endoscope. FIG. 6 is a schematic diagram for explaining of a reflection of the light output by the illuminating unit. FIG. 7 is a schematic diagram of a conventional capsule endoscope. FIG. 8 is a schematic diagram for explaining the problems in the conventional capsule endoscope.

FIG. 1 is a side view of an internal structure of a capsule endoscope 10 according to an embodiment of the present invention. The capsule endoscope 10 includes a watertight capsule casing 14. The capsule casing 14 includes a capsule trunk 22 that houses an image capturing unit 11 that captures images of an inside of an abdominal cavity, an illuminating unit 12 that outputs a light to illuminate the inside of the abdominal cavity, a power-supply unit 13 that supplies an electric power to both the image capturing unit 11 and the illuminating unit 12.

The capsule casing 14 has a front cover 20 with a window 20a that allows the light L output from the illuminating unit 12 to pass through but does not allow a reflected light of the light L to reach to the image capturing unit 11. A rear cover, if required, may be provided to the capsule trunk 22. It is assumed that, the rear cover is provided integrally with the capsule trunk and is flat, however, the rear cover may be dome-shaped.

As shown in FIG. 3, the front cover 20 may be divided into a flat window 20a and a convex window 20b. The flat window 20a is for passing the light L and the convex window 20b for the image capturing unit 11 to capture images. The whole of the front cover 20 is transparent.

The image capturing unit 11 is installed on an image capturing substrate 24. The image capturing unit 11 includes a solid-state image sensor 25 and an image forming lens 26. The solid-state image sensor 25 is, for example, a charged couple device (CCD), and captures images in the range that is illuminated by the light L. The image forming lens 26 includes a fixed lens 26a which forms an image of an object on the solid-state image sensor 25 and a movable lens 26b. The image forming lens 26 forms a sharp image by being controlled by a focusing unit 28. The focusing unit 28 includes a fixing frame to firmly hold the fix lens 26a and a movable frame to movably hold the movable lens 26b.

The image capturing unit 11 is not limited to the CCD, but may be a complementary metal-oxide semiconductor (CMOS).

The illuminating unit 12 is provided on an illuminating substrate 30 is, for example, a light emitting diode (LED). A plurality of the illuminating units 12 (four in the present embodiment) are disposed around the image forming lens 26.

The power-supply unit 13 is installed on a power-supply substrate 32 that has an internal switch 31. A button battery cell (hereinafter, “button cell”) 33, for example, is used as a source of the power supply. It is assumed here that the button cell is a silver-oxide cell; however, the button cell may be a rechargeable cell, a dynamo cell, and the like.

It is assumed here that the internal switch 31 is a magnetic switch that can be made ON/OFF using a magnet.

A wireless unit 42, which includes an antenna etc., for performing wireless communication with outside is installed on a wireless substrate 41 and performs communication with the outside according to the requirement.

A signal-processing and control unit 43 that processes and controls the various units mentioned above is installed on the image capturing substrate 24 and executes various processes in the capsule endoscope.

The signal-processing and control unit 43 includes an image-signal processing function of image-data generation etc. which includes correlated double sampling (CDS), for example, a transmission-signal generating function to perform mixing of an image signal and a synchronization signal (in a case of analog transmission) and addition of a mistake-correction sign (in a case of digital transmission), a modulation function to convert in cooperation with a modulator, to a phase-shift keying (PSK) modulation, a minimum-shift keying (MSK) modulation, a Gaussian minimum-shift keying (GMSK) modulation, a quadrature minimum-shift keying (QMSK) modulation, and an amplitude-shift keying (ASK) modulation format, for example, a power-supply control function to control the power supply according to ON-OFF operation of a switch, a timing-generator (TG) function to control a driver circuit like an LED driver circuit, a storage function to store various data like parameters of line and frame etc. and performs various signal processing and controls.

The signal processing may include image-data correction (white balance (WB) correction, γ correction, color processing, automatic gain control (AGC) etc.), analog-digital conversion (ADC), automatic exposure control function (AE), and the like.

FIG. 2 is a schematic diagram of the capsule endoscope system 50 according to the present embodiment. The capsule endoscope system 50 uses the capsule endoscope 10 to check a patient.

The capsule endoscope system 50 includes, for example, the capsule endoscope 10 and its package 51, a jacket 53 that is to be worn by a patient 52, a detachable receiver 54 that can be detachably attached to the jacket 53, and a work station 55 that processes information which is received in the receiver 54.

Antennas 56a, 56b, 56c, and 56d which catch electric waves of image signals transmitted from the wireless unit 42 of the capsule endoscope 10 are installed in the jacket 53 and are provided to enable wireless communication or wired communication by a cable with the receiver 54. Further, the number of antennas installed in the jacket 53 is not limited to four and would be more so that the electric waves from the capsule endoscope 10 can be received properly.

The receiver 54 includes a display 57 that displays information necessary for observation (examination) and an input section 58 to input information necessary for observation (examination). Moreover, a CF (compact flash (registered trademark)) memory 59 that stores image data, is detachably mounted on the receiver 54. Further, the receiver 54 is provided with a power-supply unit 60 that can supply power even while carrying and a signal processing and control section 61 that performs processing required for observation (examination). A dry battery cell, a lithium-ion secondary battery cell, nickel-hydrogen battery cell etc. are examples of the power-supply unit 60 and it may be a rechargeable battery cell as well.

The work station 55 has processing functions to perform diagnosis based on images of internal organs in a body of a patient which a doctor or a nurse has captured by the capsule endoscope 10. This work station 55 is provided with a CF memory reader/writer 61. It is not shown in the diagram but the receiver 54 and the CF memory reader/writer 61 have interfaces that can be connected to enable communication, and read and write the CF memory 59.

Moreover, the work station 55 has a communication function for connecting to a network and via this network a medical examination result of the patient is stored in a database. Further, the work station 55 has a display 62 and inputs the captured image data of inside of the patient's body from the receiver 54 and displays images of internal organs etc. on the display 62.

When carrying out the examination, the capsule endoscope 10 is taken out from the package 51 and the patient 52 swallows the capsule endoscope 10. The capsule endoscope 10 passes through esophagus of the patient, advances to an abdominal cavity due to peristalsis of an alimentary canal cavity and captures images inside the abdominal cavity one after another.

The capsule endoscope 10 transmits, continuously or intermittently, the electric signals corresponding to the captured images via the wireless unit 42. The antennas 56a to 56d receive those electric signals and transmit them to the receiver 54.

The receiver 54 stores the electric signals in the CF memory 59 in the form of captured image data. The operation of the receiver 54 is not synchronized with the start of image capturing of the capsule endoscope 10, but, the start and the end of receiving are controlled by an operation of the input section 58. Moreover, the captured image data may be still-image data that is captured at a plurality of frames per second for displaying them as moving images or may be normal video-image data.

When the observation (examination) of the patient 52 by the capsule endoscope 10 is completed, the CF memory 59 is taken out of the receiver 54 and inserted into the CF memory reader/writer 61. The data in the CF memory 59 is transferred to the work station 55. In the work station 55, the data for each patient is stored and managed separately.

Thus, the captured image data of the inside of the abdominal cavity that is captured by the capsule endoscope 10 and stored by the receiver 54 is displayed as image data by the display 62 in the work station 55. This enables to acquire data useful for physiological research and to make a diagnosis of a physical change caused by a disease of the entire alimentary canal in the human body including internal organs which are not accessible (like a small intestine) by an ultrasonic probe, endoscope etc.

The front cover is described in detail with reference to FIG. 1, FIG. 3, and FIG. 6.

The front cover 20 covers the image capturing unit 11 and the illuminating unit 12. Moreover, the front cover 20 has the window 20a and the window 20b. In the embodiment shown in FIG. 1 the entire surface of the front cover is flat, while in the embodiment shown in FIG. 3 the window 20b of the front cover 20 is convex.

In both of the embodiments previously described, the window 20a through which the light L passes is flat to prevent reflection of the light L.

Because the window 20a is flat, the light L passes at almost right angle to the window 20a and no light is reflected toward the image capturing unit 11. The light incident becomes the outgoing light DL that illuminates the abdominal cavity and there is no effect on the image capturing unit 11 due to the reflection.

Moreover, as the front cover 20 shown in FIG. 3 and FIG. 4, a central part is protruded out to form a convex part 21a and a part other than the convex part is made to be a window 21b for outgoing illuminating light. This form of the front cover 20 makes it easy for the patient to swallow the capsule endoscope 10.

By making the front cover 20 as in the present embodiment, since a central optical axis of the light L from the illuminating unit 12 and the window 20b for image capturing are at right angles to each other as shown in FIG. 5, the reflection in the image forming lens 26 in the image capturing unit 11 is prevented as shown in FIG. 6.

In other words, the light L is incident straight on the window 20a and passes through easily. Thus, since the light L is not reflected to the image capturing unit 11, flare etc. is prevented.

Furthermore, as shown in FIG. 6, regarding a distance D between the window 20b and the illuminating unit 12, a range of 0.01 mm to 3 mm is desirable, and a range of 0.1 mm to 2 mm is more desirable. If the distance D is more than 3 mm, for example, in a case of an LED, since emitted radially, it is not favorable.

Moreover, it is desirable that a connecting portion with the capsule trunk 22 of the front cover is in the form of letter R. This is because the curve surface eases swallowing by the patient.

Furthermore, when a distance between illuminating unit 12 and the flat window 20a is short, an area of the flat window 20a may be slightly less than an area of a surface of the illuminating unit 12 and when the distance between the two becomes longer, at least the area of the flat window 20a is required to be kept roughly the same as the area of the surface of the illuminating unit 12. This is because as the distance between the illuminating unit 12 and the flat window 20a for illuminating light becomes longer, the reflected light tend to reach towards the image capturing unit 11.

According to the present embodiment, since the window for illuminating the light is made such that illuminating light cannot be reflected easily to an image capturing unit, it is possible to prevent reflection to the image capturing unit thereby enabling to obtain clear images.

Moreover, by making a capsule endoscope system that includes the capsule endoscope, it is possible to make an examination with high accuracy.

Although the invention has been described with respect to a specific embodiment for a complete and clear disclosure, the appended claims are not to be thus limited but are to be construed as embodying all modifications and alternative constructions that may occur to one skilled in the art which fairly fall within the basic teaching herein set forth.

What is claimed is:

1. A capsule endoscope comprising:
an image capturing unit that has an image capturing section which can capture images of an inside of an abdominal cavity;
an illuminating unit that outputs light and illuminates the inside of the abdominal cavity with the light;
a power-supply unit that supplies power to the image capturing unit and the illuminating unit;
a front cover that covers the image capturing unit and the illuminating unit and is provided with an illuminating window that lets the light output by the illuminating unit to pass through and that makes it hard for the light output by the illuminating unit to be reflected toward the image capturing unit; and
a capsule casing that is attached to the front cover such that there is a watertight space inside, wherein the image capturing unit, the illuminating unit, and the power-supply unit are housed in the watertight space.

2. The capsule endoscope according to claim 1, wherein the image capturing unit and the illuminating window are separated apart from each other by such a distance that it is hard for a light reflected from the illuminating window to reach the image capturing unit.

3. The capsule endoscope according to claim 2, wherein the image capturing unit and the illuminating window are separated apart from each other by 2 millimeter or less.

4. The capsule endoscope according to claim 2, wherein the illuminating unit and the image capturing unit are separated apart from each other by 1 millimeter or less.

5. The capsule endoscope according to claim 1, wherein the illuminating window substantially covers a light emitting surface of the illuminating unit completely from front.

6. The capsule endoscope according to claim 1, wherein the illuminating window is disposed in a direction at right angles to a central optical axis of the light output by the illuminating unit.

7. The capsule endoscope according to claim 1, wherein the illuminating window is made of either a resin or glass.

8. The capsule endoscope according to claim 1, wherein a central portion of the front cover other than the illuminating window is convex.

9. The capsule endoscope according to claim 1, wherein at least two illuminating units are disposed around the image capturing unit.

10. The capsule endoscope according to claim 1, further comprising a communication unit that transmits to outside image information acquired by the image capturing unit.

11. A capsule endoscope system comprising:
a capsule endoscope including
an image capturing unit that has an image capturing section which can capture images of an inside of an abdominal cavity;
an illuminating unit that outputs light and illuminates the inside of the abdominal cavity with the light;
a power-supply unit that supplies power to the image capturing unit and the illuminating unit;
a front cover that covers the image capturing unit and the illuminating unit and is provided with an illuminating window that lets the light output by the illuminating unit to pass through and that makes it hard for the light output by the illuminating unit to be reflected toward the image capturing unit;
a communication unit that transmits to outside image information acquired by the image capturing unit; and
a capsule casing that is attached to the front cover such that there is a watertight space inside, wherein the image capturing unit, the illuminating unit, and the power-supply unit are housed in the watertight space;
a package that covers the capsule endoscope before the capsule endoscope is used;
a receiving unit that receives the image information from the capsule endoscope; and
an information processor that processes the image information received by the receiving unit.

12. A capsule endoscope comprising:
a casing to house an illuminating unit and an image capturing unit, and a cover for the casing, the cover having
an illuminating window that has a flat surface and through which the illuminating unit illuminates a portion inside a body of a patient; and
an image capturing window through which the image capturing unit captures an image of the portion illuminated by the illuminating unit.
